(19) United States
(12) Patent Application Publication
(10) Pub. No.: US 2016/0317473 A1
(43) Pub. Date: Nov. 3, 2016

(54) METHODS OF TREATING OR PREVENTING BRAIN DAMAGE DUE TO HYPOXIC-ISCHEMIA

(71) Applicants: EMORY UNIVERSITY, Atlanta, GA (US); CHILDREN'S HEALTHCARE OF ATLANTA, INC., Atlanta, GA (US)

(72) Inventors: Chia-Yi Kuan, Marietta, GA (US); Dianer Yang, Lutherville-Timonium, MD (US); Yu-Yo Sun, Decatur, GA (US)

(21) Appl. No.: 14/716,007

(22) Filed: May 19, 2015

Related U.S. Application Data
(60) Provisional application No. 62/001,102, filed on May 21, 2014.

(51) Int. Cl. A61K 31/137; A61K 31/426

(52) U.S. Cl. CPC A61K 31/137; A61K 31/426

(57) ABSTRACT

This disclosure relates to managing brain injury. In certain embodiments, this disclosure relates to methods comprising administering an effective amount of an inhibitor of sphingosine-1-phoshate signaling, an inhibitor of retinoic-acid-receptor-related orphan receptors α or γ, and/or an inhibitor of tPA microglial activation to a subject in need thereof. Typically, the subject is a neonate or a neonate born prematurely.

### CROSS REFERENCE TO RELATED APPLICATIONS

This Application claims priority to U.S. Provisional Application No. 62/001,102 filed May 21, 2014, hereby incorporated by reference in its entirety.

### STATEMENT REGARDING FEDERALLY FUNDED RESEARCH

This invention was made with government support under Grant NS074559 awarded by the National Institutes of Health. The government has certain rights in the invention.

### BACKGROUND

Perinatal asphyxia is caused by a lack of oxygen (hypoxic) and blood (ischemia) in the brain of a newborn infant typically experienced during the birthing process. Hypoxicischemia in the brain is concerning because it can cause death, or the neonate may survive having abnormal brain development (encephalopathy) manifested as a mental condition, such as cerebral palsy. Perinatal asphyxia happens more often in premature neonates who typically present symptoms such as a combination of abnormal color (cyanosis), perfusion, responsiveness, muscle tone, and respiratory effort. If resuscitation is successful, the infant is usually transferred to a neonatal intensive care unit wherein moderate induced hypothermia (32.0-34.0° C.) is a typical treatment. Therapeutic hypothermia is also recommended for term infants exhibiting symptoms that can lead to encephalopathy. Shankaran, Curr Treat Options Neurol, 2012, 14(6):608-19. A significant number of infants treated with hypothermia suffer significant neurological disabilities. Thus, there is a need to identify improved treatment methods.

Intrauterine infection (chorioamnionitis) of the pregnant mother is a high risk factor for cerebral palsy in term and near-term neonates. Studies indicate that a combination of perinatal infection and hypoxic-ischemic (HI) insult also causes greater brain injury and poorer response to therapeutic hypothermia. How remote maternal infection aggravates neonatal HI brain injury is not well understood.

Duggan et al. report intrauterine T-cell activation and increased proinflammatory cytokine concentrations in preterm infants with cerebral lesions. Lancet, 2001, 358:1699-1700.

Kallapur et al. report intra-amniotic IL-1beta induces fetal inflammation in rhesus monkeys and alters the regulatory T cell/IL-17 balance. J Immunol, 2013, 191:1102-1109. See also Korn et al., Annual review of immunology, 2009, 27, 485-517 2009.

Ghoreschi et al. report the generation of pathogenic T_{H}17 cells in the absence of TGF-beta signalling. Nature, 2010, 467:967-971.

Brinkmann et al. report fingolimod (FTY720) for use as drug to treat multiple sclerosis. Nat Rev Drug Discov, 2010, 9:883-897. Deogracias et al. report fingolimod, a sphingosine-1 phosphate receptor modulator, increases BDNF levels and improves symptoms of a mouse model of Rett syndrome. Proc Natl Acad Sci U S A, 2012, 109:14230-14235.

Yang et al. report treating neonatal hypoxic-ischemic brain injury by intranasal delivery of plasminogen activator inhibitor-1. Stroke, 2013, 44(9):2623-7.

References cited herein are not an admission of prior art.

### SUMMARY

This disclosure relates to managing brain injury. In certain embodiments, this disclosure relates to methods comprising administering an effective amount of an inhibitor of sphingosine-1-phoshate signaling, an inhibitor of retinoic-acid-receptor-related orphan receptors α or γ, and/or an inhibitor of tPA microglial activation to a subject in need thereof. Typically, the subject is a neonate or a neonate born prematurely.

In certain embodiments, the methods further comprise administering an inhibitor of sphingosine-1-phoshate signaling such as fingolimod optionally in combination with an inhibitor of retinoic-acid-receptor-related orphan receptors α or γ (RORα and RORγ) and/or a plasminogen activator inhibitor-1 or variant thereof.

In certain embodiments, the methods further comprise administering a sphingosine-1-phoshate signaling, an inhibitor of retinoic-acid-receptor-related orphan receptors α or γ, and/or an inhibitor of tPA microglial activation in combination with induced hypothermia. Typically, therapeutic hypothermia reduces the body temperature of the neonate to below 35 or 34 degrees Celsius and is continued for more than 6 hours, 24 hours, or two days.

In certain embodiments, the subject is a neonate and the mother is diagnosed with a bacterial or viral infection such as chorioamnionitis during or within one or two months of birth.

In certain embodiments, the disclosure relates to methods of treating or preventing brain injury comprising administering an inhibitor of retinoic-acid-receptor-related orphan receptors α or γt (RORα and RORγ) to a subject in need thereof.

In certain embodiments, the inhibitor of retinoic-acid-receptor-related orphan receptors α or γt is N-(5-(N-(4-(1,1,1,3,3,3-hexafluoro-2-hydroxypropan-2-yl)phenyl)sulphamoyl)-4-methylthiazol-2-yl)acetamide (SR1001), derivative, ester, or salt thereof.

### BRIEF DESCRIPTION OF THE FIGURES

FIG. 1A shows data indicating a combined infection-HI induces early-stage T_{H}17-cells in the blood and infiltration of CD43+ leukocytes in choroid plexus. The cord blood mononuclear cells of preterm neonates, with and without histological chorioamnionitis (n=8-9 for each group), were compared for the mRNA levels of IL-23 receptor (normalized to CD3). Mononuclear cells in preterm neonates with severe chorioamnionitis had significant increase (p<0.05) of IL-23R transcripts.

FIG. 1B shows data on the peripheral blood mononuclear cells of 7-day-old rat pups at 4 h after 0.3 mg/kg LPS challenge (n=5), pure-HI insult (n=6), and the combined LPS/HI insult with saline (PBS, n=6) or FTY720 treatment (n=7) were collected to compare the IL-23R mRNA levels (normalized to actin). LPS/HI injury significantly increased the expression of IL-23R mRNA, which was blocked by the FTY720 treatment.

FIG. 1C-1 and FIG. 1C-2 shows flow cytometry indicating preferential reduction of CD4+ T-cells in peripheral blood mononuclear cells by the FTY720 treatment at 16 h recovery (13%, n=5), when compared to untouched (61%, n=3), low-dose LPS exposed (72%, n=3), pure-HI injured (66%, n=5), or LPS/HI-injured siblings (69%, n=5). Flow cytometry also showed a small increase of CD4+ IL17A+ T-cells after pure-HI (2.17%) and LPS/HI injury (4.92%), when compared to untouched (0.64%), low-dose LPS-exposed (1.04%), and LPS/HI-challenged but receiving the FTY720 treatment (0.12%) rat pups.

FIG. 1D shows immunostaining indicates an intense ribbon-like anti-CD43 (leukosialin, W3/13) reactivity in the choroid plexus of unchallenged P7 rat pups, which was reduced at 4 h after LPS exposure (0.3 mg/kg) and in the contralateral hemisphere after LPS/HI insult. In contrast, the ribbon-like CD43 immunoreactivity was greatly reduced in the ipsilateral choroid plexus at 4 h following pure-HI or LPS/HI insult. CD43+ cells (arrows) scattered to the nearby subcortical white matter were also detected in the ipsilateral LPS/HI-injured choroid plexus. The reduction of ribbon-like CD43-immunoreactivity in the LPS/HI-injured plexus was less pronounced following the FTY720 treatment (n=5 for each condition). Scale bar: 30 μm.

FIG. 2A shows data indicating CD43+ leukocytes enter neonatal brains after LPS/Hi injury in distinct spatiotemporal routes. Immunocytochemistry was used to examine the influx of CD43+ leukocytes and their relationship to Ibal+ microglia/monocytes at 4 and 24 h following LPS/HI injury and the saline-versus FTY720 treatment. At 4 h post-LPS/HI injury, CD43+ leukocytes mingled with Ibal+ ameboid microglia/monocytes was detected in the ipsilateral subcortical white-matter (WM), but not in the contralateral hemisphere or after the FTY720 treatment. CD43+ leukocytes were only accumulated in the ipsilateral leptomeningeal space of LPS/HI-injured and saline-treated rat pups at 24 h, but not 4 h recovery. The FTY720 treatment also prevented the accumulation of CD43+ leukocytes in the leptomeningeal space. At 24 h recovery, CD43+ leukocytes mingled with round, activated Ibal+ microglia/monocytes were detected in the ipsilateral cortical gray matter (GM) of LPS/HI-injured rat pups. In contrast, CD43+ leukocytes were absent in the contralateral hemisphere or in pups that received FTY720. The NF-κB EMSA with the rat brain nuclear extract collected at 4 h recovery from LPS/HI insult and the saline-versus-FTY720 treatment. The FTY720 treatment prevented the high-order DNA-protein complex using a specific NF-κB probe (shown are the responses of 3 animals for each treatment). Mutant NF-κB probe was used as negative controls.

FIG. 2B shows data where LPS/HI insult caused reduction of the cytoplasmic IκBα in the ipsilateral hemisphere (asterisks; right, R), which was also averted by the FTY720 treatment (shown are typical results in two subjects).

FIG. 3A shows data indicating FTY720 prevents LPS/HI-induced accumulation of CD4+ IL-17A+ lymphocytes and neuroinflammation in neonatal brains. Flow cytometry showed significant increase of CD4+ IL-17A+ cells in the brain of LPS/HI-injured rats (n=7) at 24 h recovery, which was reduced by the FTY720 treatment (n=8). In contrast, neither untouched (n=6), 0.3 mg/kg LPS challenged (n=4), or pure-HI injured rats (n=5) showed >3% of CD4+ IL-17A+ T-cells among all CD4+ lymphocytes.

FIG. 3B shows data indicating the combined LPS/HI injury caused significantly increased RORc, IL-23R, and IL-17A mRNA, as well as a trend of increase in IL-22 mRNA (all T_{H}17 markers) than pure-HI injury (n=4 for each). The induction of Tbx21/T-bet (a T_{H}1 and mixed T_{H}1/T_{H}17 cell marker), IFNγ (a T_{H}1 cell marker), or Gata3 and IL-13 mRNA (T_{H}2 cell markers) was similar between pure-HI and LPS/HI insults. The FTY720 treatment significantly decreased the LPS/HI-induced expression of RORc, IL-23 receptor, IL-17A, and Tbx21/T-bet mRNA, but not in IFNγ, Gata3 or IL-13 mRNA (n=4).

FIG. 3C shows data indicating cytokine array showed 9.7-fold increase of the brain MCP1 (Monocyte Chemoattractant Protein-1) protein at 24 h after the LPS/HI insult, which was reduced to 3-fold in FTY720-treated rats (n=4 for each).

FIG. 3D shows data indicating ELISA also showed significant attenuation of the LPS/HI-induced MCP1 expression in the brain, but not the plasma, of FTY720-treated animals (n=4 for each).

FIG. 3E shows data indicating that FTY720 treatment also markedly reduced the post-LPS/HI induction of TSPO (Translocation protein of 18 kDa, a marker for activated microglia) mRNA (n=4 for each).

FIG. 4A shows data indicating FTY720 exerts no direct inhibition of microglial activation. FTY720 (1 μg/g, IP) was unable to prevent the induction of MCP1 after intracerebroventricular (ICV) injection of LPS (2 μg) in P7 rats, a model of direct microglial activation.

FIG. 4B shows data for visualization of microglia and monocytes in P10 CX3CR1-GFP; CCR2-RFP mice and 24 h after ICV injection of LPS (1 μg) with saline or FTY720 treatment (1 μg/g). In untouched mice, few GFP+ microglial processes extended into the pyramidal cell layer in the hippocampal (CA), and almost no RFP+ monocytes were detected in the brain. After ICV-LPS injection with either saline or FTY720 treatment, hypertrophic microglial processes extended into the pyramidal cell layer and a large number of monocytes were found in the brain (n=3 for each).

FIG. 4C shows data indicating the application of phospho-FTY720 (2-10 μM) failed to prevent LPS (5 ng/m1)-induced iNOS and COX2 expression in immortalized microglia SM826 cells (repeated 3 times).

FIG. 4D shows data indicating phospho-FTY720 failed to attenuate the LPS-mediated induction of inflammatory cytokines, including IL-1β and IL-6, in SM826 cells (repeated 3 times). E, RT-qPCR analysis showed high expression of the SIP receptor-1 (S1PR1) and -2 (S1PR2) isoforms in neonatal rat microglia. Previous study showed that phospho-FTY720 lacks binding affinity to the S1PR2 isoform (Mandala et al., 2002).

FIG. 4E shows data indicating neither phospho-FTY720 nor sphingosine-1-phosphate (S1P) prevented LPS-induced IL-23 mRNA in SM826 microglial cells (shown are the mean and standard deviation of three experiments).

FIG. 4F shows data indicating immunoblot analysis of the effect of S1P and Tat-NBD peptide (5, 20 μM) on LPS (5 ng/ml)-induced NFKB signaling activation in SM826 microglial cells. The Tat-NBD peptide showed dose-dependent attenuation of IκBα degradation in the cytosol and nuclear accumulation of NFκB/RelA at 30 min after LPS-exposure, while S1P lacked this effect (shown are the typical result in three experiments).

FIG. 5A shows data indicating FTY720 markedly reduces LPS/HI brain injury, but is ineffective against pure HI injury. Because 80 min pure-HI insult caused little brain damage, a longer duration of pure-HI (90 min) was used to compare with the combined LPS/HI (80 min) insult. Representative brain photographs of P8 rats that were treated by the indicated condition 24 h earlier and received IP injection of NaF at 2 h prior to transcardial perfusion; the residual NaF-fluorescence in the HI-injured hemisphere (asterisks) indicates BBB damage and increased permeability. Quantification of NaF fluorescence in the LPS-, HI-, or LPS/HI-treated hemisphere compared to untouched (UN) brains. LPS/HI injury markedly induced BBB permeability, which was significantly reduced by the FTY720 treatment (n=3 for untouched, 4 for LPS-alone, 5 for HI, LPS/HI-PBS, and LPS/HI-FTY720 groups).

FIG. 5B shows data on MMP zymogram of the ipsilateral rat cortical hemisphere collected at 24 h after the indicated treatment. Shown are three typical samples in each condition. Asterisk: non-specific band. Note the induction of MMP9 by HI or LPS/HI insult, additional processing of MMP9 in LPS/HI insult, and the absence of MMP9 induction in FTY720-treated animals.

FIG. 5C show data on MMP zymogram of rat brains subjected to HI injury and the saline-versus FTY720 treatment at 24 h recovery. Shown are two representative samples in each treatment. The FTY720 therapy failed to prevent HI-induced MMP9 activation in the ipsilateral (right, R) cortical hemisphere.

FIG. 6A shows data on quantification indicating significant preservation of ADC signals in the cerebral cortex of FTY720-treated rat pups (n=8), when compared to those receiving the saline treatment (n=9). FTY720 protects white-matter and motor development after neonatal LPS/HI injury. Manganese-enhanced MRI (MEMRI) comparison at 24 h recovery indicated the disappearance of cytoarchitectural distinctions in the ipsilateral hemisphere (asterisk) in saline-treated, but not FTY720-treated rats (n=4-5 for each group). Comparison of apparent diffusion coefficient (ADC) at 24 h after LPS/HI indicates the reduction of ADC signals in the ipsilateral hemisphere in saline-treated animals (asterisk). Sodium MRI acquisition and quantification showed spatial correlation between increased sodium concentration and reduced ADC signal.

FIG. 6B show data on a comparison of the latency to fall (seconds) on rotarods in untouched, LPS/HI-injured and saline- or FTY720-treated rat pups at 24 d of age (n=3 for untouched, 8 each for saline- and FTY720-treated animals).

FIG. 7 illustrates a proposed mechanism of brain injury due to HI. It is not intended that embodiments of this disclosure be limited by the proposed mechanism. HI-induced parenchymal tPA and infection/HI-triggered brain influx of early-onset T_{H} 17 cells converge to cause greater microglia activation, which feeds back to promote T_{H}17 cell maturation. This hypothesis suggests three therapeutic targets: the S 1P (sphingosine-1-phosphate) signaling for lymphocyte trafficking, the RORc transcriptional factor for the synthesis of toxic Th17-cytokines, and tPA for enhanced microglial activation and tissue proteolysis.

FIG. 8A illustrates the structure of SR1001.

FIG. 8B shows data on tissue loss with SR1001.

FIG. 8C shows data on T_{H}17 differentiation.

FIG. 8D shows data on microglia.

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise.

In this specification and in the claims that follow, reference will be made to a number of terms that shall be defined to have the following meanings unless a contrary intention is apparent.

Prior to describing the various embodiments, the following definitions are provided and should be used unless otherwise indicated.

“Subject” means any animal, but is preferably a mammal, such as, for example, a human, monkey, mouse, or rabbit.

As used herein, the terms “prevent” and “preventing” include the prevention of the recurrence, spread or onset. It is not intended that the present disclosure be limited to complete prevention. In some embodiments, the onset is delayed, or the severity is reduced.

As used herein, the terms “treat” and “treating” are not limited to the case where the subject (e.g. patient) is cured and the disease is eradicated. Rather, embodiments of the present disclosure also contemplate treatment that merely reduces symptoms, and/or delays disease progression.

As used herein, the term “combination with” when used to describe administration with an additional treatment means that the agent may be administered prior to, together with, or after the additional treatment, or a combination thereof.

As used herein, the term “derivative” refers to a structurally similar compound that retains sufficient functional attributes of the identified analogue. The derivative may be structurally similar because it is lacking one or more atoms, substituted, a salt, in different hydration/oxidation states, or because one or more atoms within the molecule are switched, such as, but not limited to, replacing a oxygen atom with a sulfur atom or replacing an amino group with a hydroxyl group. The derivative may be a prodrug. Derivatives may be prepare by any variety of synthetic methods or appropriate adaptations presented in synthetic or organic chemistry text books, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

The term “substituted” refers to a molecule wherein at least one hydrogen atom is replaced with a substituent. When substituted, one or more of the groups are “substituents.” The molecule may be multiply substituted. In the case of an oxo substituent (“═O”), two hydrogen atoms are replaced. Example substituents within this context may include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRaRb, —NRaC(═O)Rb, —NRaC(═O)NRaNRb, —NRaC(═O)ORb, —NRaSO2Rb, —C(═O)Ra, —C(═O)ORa, —C(═O)NRaRb, —OC(═O)NRaRb, —ORa, —SRa, —SORa, —S(═O)2Ra, —OS(═O)2Ra and —S(═O)2ORa. Ra and Rb in this context may be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, and heteroarylalkyl.

As used herein, the term “plasminogen activator inhibitor-1” refers to a natural or synthetic polypeptide comprising SEQ ID NO:1 VHHPPSYVAHLASDFGVRVFQ QVAQASKDRNVVFSPYGVASVLAMLQLTTGGETQQQIQAAMGFKIDDKGMAPAL RHLYKELMGPWNKDEISTTDAIFVQRDLKLVQGFMPHFFRLFRSTVKQVDFSEVER ARFIINDWVKTHTKGMISNLLGKGAVDQLTRLVLVNALYFNGQWKTPFPDSSTHR RLFHKSDGSTVSVPMMAQTNKFNYTEFTTPDGHYYDILELPYHGDTLSMFIAAPYE KEVPLSALTNILSAQLISHWKGNMTRLPRLLVLPKFSLETEVDLRKPLENLGMTDM FRQFQADFTSLSDQEPLHVAQALQKVKIEVNESGTVASSSTAVIVSARMAPEEIIMD RPFLFVVRHNPTGTVLFMGQVMEP or variants have greater than 90, 95, 96, 97, 98, or 99 identity or similarity thereto.

In certain embodiments, sequence “identity” refers to the number of exactly matching amino acids (expressed as a percentage) in a sequence alignment between two sequences of the alignment calculated using the number of identical positions divided by the greater of the shortest sequence or the number of equivalent positions excluding overhangs wherein internal gaps are counted as an equivalent position. For example the polypeptides GGGGGG and GGGGT have a sequence identity of 4 out of 5 or 80%. For example, the polypeptides GGGPPP and GGGAPPP have a sequence identity of 6 out of 7 or 85%. In certain embodiments, any recitation of sequence identity expressed herein may be substituted for sequence similarity. Percent “similarity” is used to quantify the similarity between two sequences of the alignment. This method is identical to determining the identity except that certain amino acids do not have to be identical to have a match. Amino acids are classified as matches if they are among a group with similar properties according to the following amino acid groups: Aromatic-F Y W; hydrophobic-A V I L; Charged positive: R K H; Charged negative-D E; Polar-S T N Q.

Contemplated variants include any having one, two, three, four, or five the following substitutions to SEQ ID NO: 1, substitute P36 to S, T49 A, A12 to V, K141 to Q, I91 to L, T93 to S, A318 to S, V334 to E, M 354 to I, N150 to H, M45 to K, Q55 to L, Q123 to R, T333 to S, G84 to R, F236 to I, E212 to V, A156 to S, V343 to A, E327 to V, L160 to Q, M235 to L, F372 to I, T232 to S, 1253 to T, R271 to C, S331 to G, S13 to K, or D14 to T. See Berkenpas et al., EMBO J. 1995, 14(13):2969-77.

Blocking Lymphocyte Trafficking with FTY720 Prevents Inflammation-Sensitized Hypoxic-Ischemic Brain Injury in Newborns

It is not well understood how remote maternal infection aggravates neonatal hypoxic-ischemic (HI) brain injury since newborns are traditionally considered immunodeficient, lacking the adaptive immunity to initiate systemic anti-pathogen responses. It is thought that chorioamnionitis induces circulating CD45RO+ effector/memory T-cells associated with brain damage in preterm neonates. Because the traditional T_{H}1 or T_{H}2 cells are yet to mature in neonates, the early-onset active T-cells following infection/HI injury may be that of T_{H}17-like lymphocytes that have a high propensity to induce autoimmune and inflammatory disorders. Ghoreschi et al. report the generation of pathogenic T_{H}17 cells in the absence of TGF-beta signaling. Nature 467:967-971.

Inflammation-sensitized HI brain injury is less responsive to therapeutic hypothermia than pure-HI injury in neonates. Studies herein indicate that inflammation-sensitized HI triggers early onset of T_{H}17/IL-17-mediated immune responses to damage neonatal brains, which can be ameliorated by blocking lymphocyte trafficking with fingolimod (FTY720), an oral medicine that is in clinical use for treating multiple sclerosis.

In support of divergent pathological mechanisms, past studies have shown that in LPS-sensitized HI injury, the brain innate immune responses and NF-κB activity are triggered at as early as 4 h recovery. In contrast, microglia are activated secondary to tissue damage in a delayed manner following pure-HI insult. Further, gene deletion of MyD88 or pharmacological inhibition of the NF-κB pathway markedly reduces LPS-sensitized neonatal HI brain injury, but confers little to no protection against pure-HI injury. Studies herein suggest that the dual hit of systemic inflammation and HI uniquely induces the influx of early-stage T_{H}17 cells to coordinate neuroinflammation, which can be successfully blocked by FTY720 treatment.

Although it is not intended that embodiments of the disclosure be limited by any particular mechanism, it is thought that FTY720 is able to sequester lymphocytes in lymph nodes and block their trafficking to the target organs. Consistent with this mechanism, post-LPS/HI administration of FTY720 markedly reduced systemic CD4+ T-cells, rapid influx of CD43+ leukocytes in the choroid plexus, acute activation of the NF-κB signaling pathway, and accumulation of CD4+ IL-17A+T-cells in the neonatal brain. FTY720 (and its active metabolite pFTY720) neither opposes pure-HI brain injury nor directly inhibits microglial activation. These paradoxical “negative” findings are in agreement with recent reports showing that FTY720 neither reduces acute brain infarction nor exerts direct neuroprotection, although it reduces the influx of lymphocytes in cerebral ischemia. Further, genetic ablation of IL-17 fails to reduce acute cerebral ischemic injury.

Studies indicate that the combined inflammation/HI injury does not induce a large increase of systemic T_{H}17 cells at 16-24 h recovery, while significant increase of CD4+ IL-17A+T-cells are only present in neonate brains. This restricted distribution pattern suggests that full maturation of T_{H}17 cells may require stimulation by activated microglia in the brain. This scenario is in accord with a study showing that upon LPS stimulation, neonatal T cells up-regulate a functional IL-23 receptor to become early-stage T_{H}17 cells and respond to IL-23 secreted by tissue-specific dendritic cells to become effector T_{H}17 lymphocytes.

Similarly, rapid induction of IL-23R mRNAs was detected in the peripheral blood mononuclear cells and expression of IL-23 was detected by LPS-stimulated microglia. Together, these results suggest that activated microglia or monocytes in LPS/HI-injured neonatal brains may secrete IL-23 to promote full maturation of T_{H}17 effector cells.

FTY720 is a promising treatment of inflammation-sensitized HI brain injury in term or near-term neonates. While FTY720 is an immune-modulator, it does not promote post-stroke bacterial infections, and experiments herein indicate that it does not impair neonatal development or worsen pure-HI brain injury. Thus, FTY720 is a useful adjuvant to therapeutic hypothermia when prenatal infection of asphyxiated infants is suspected.

Although it is not intended that embodiments of this disclosure are limited by any particular mechanism, it is thought that pure HI injury activates microglia secondary to neuronal cell injury by innate immune response—peripheral inflammation magnifies and accelerates microglial activation leading to greater brain damage. A combination of peripheral inflammation and HI stimulates the influx of early-onset T_{H}17 cells into the neonatal brain. T_{H}17-released cytokines and HI-induced parenchymal tissue plasminogen activator (tPA) enhance microglial activation, which not only causes greater injury but also reciprocates to induce full maturation of T_{H}17 cells (FIG. 7). Thus, infection-sensitized neonatal HI brain injury can be mitigated by systemic administration of FTY720 (a drug that blocks S1P signaling and lymphocyte trafficking) or SR1001 (a small-molecule inhibitor of RORc that is important for T_{H}17 cell differentiation) or CNS-targeted CPAI therapy (a stable-mutant plasminogen activator inhibitor-1 [PAH] to oppose tPA). See Berkenpas et al. EMBO J. 1995, 14(13): 2969-2977 and Yang et al., Stroke. 2013, 44(9):2623-7.

### Methods of Use

In certain embodiments, this disclosure relates to methods comprising administering an effective amount of an inhibitor of sphingosine-1-phoshate signaling, an inhibitor of retinoic-acid-receptor-related orphan receptors α or γ, and/or an inhibitor of tPA microglial activation to a neonate in need thereof.

In certain embodiments the inhibitor of sphingosine-1-phoshate signaling is a S1P-specific humanized antibody, fingolimod (FTY720 or 2-amino-2-[2-(4-octylphenyl)ethyl]propane-1,3-diol), (2R,3S ,4E)-N-methyl-5-(4′-pentylphenyl)-2-aminopent-4-ene-1,3-diol , Safingol, (L-threo-dihydrosphingosine), 2-(p-hydroxyanilino)-4-(p-chlorophenyl)thiazole, N,N-dimethylsphingosine (DMS), (E)-1-(4-((1R,2S,3R)-1,2,3,4-tetrahydroxybutyl)-1H-imidazol-2-yl)ethanone oxime (LX2931) and (1R,2S,3R)-1-(2-(isoxazol-3-yl)-1H-imidazol-4-yl)butane-1,2,3,4-tetraol (LX2932), resveratrol, 3-(4-chlorophenyl)-adamantane-1-carboxylic acid (pyridin-4-ylmethyl)amide, N-(1-Carbamimidoylcyclopropyl)-4-dodecylbenzamide, N-(1-Amino-1-iminopropan-2-yl)-4-octylbenzamide, derivative, ester, or salt thereof.

In certain embodiments, the inhibitor of retinoic-acid-receptor-related orphan receptors α or γt is N-(5-(N-(4-(1,1,1,3,3,3-hexafluoro-2-hydroxypropan-2-yl)phenyl)sulphamoyl)-4-methylthiazol-2-yl)acetamide (SR1001), 1,1,1,3,3,3-hexafluoro-2-(2-fluoro-4′-((4-(pyridin-4-ylmethyl)piperazin-1-yl)methyl)-[1,1′-biphenyl]-4-yl)propan-2-ol (SR2211), 1,1,1,3,3,3-Hexafluoro-2-(4′-((1-(acetyl) piperazine) methyl)-[1,1′-biphenyl]-4-yl)propan-2-ol (SR1555), 3-(Benzo[d][1,3]dioxol-5-yl)-1-cis-3,5-dimethylpiperidin-1-yl)-3-(2-hydroxy-4,6-dimethoxyphenyl)propan-1-one (ML209), N-(5-benzoyl-4-phenylthiazol-2-yl)-2-(4-(ethylsulfonyl)phenyl)acetamide, N-(4-(1,1,1,3,3,3-hexafluoro-2-hydroxypropan-2-yl)phenyl)-N-(2,2,2-trifluoroethyl)benzenesulfonamide (T0901317), 24R-hydroxycholesterol, 24,25-epoxy-cholesterol, 24S-hydroxycholesterol, 7-ketocholesterol, digoxin, ursolic acid, or salt or derivative or ester thereof.

In certain embodiments, an inhibitor of tPA microglial activation is plasminogen activator inhibitor-1 or variants thereof.

In certain embodiments, this disclosure relates to methods of treating or preventing brain injury comprising administering an effective amount of a sphingosine-1-phoshate signaling, an inhibitor of retinoic-acid-receptor-related orphan receptors α or γ, and/or an inhibitor of tPA microglial activation to a subject in need thereof. Typically, the subject is a neonate or a neonate born prematurely. In certain embodiments, the neonate is born more than one, two, three, or four weeks, premature, or of a birth of less than 37 weeks gestational age, the actual beginning of the last menstrual period. In certain embodiments, the neonate is considered premature, e.g., more than two weeks and born after 22 or 24 weeks of gestation. Ovulation occurs on average 14.6 days after the beginning of the preceding menstruation (LMP).

In certain embodiments, the methods further comprise administering fingolimod optionally in combination with an inhibitor of retinoic-acid-receptor-related orphan receptors α or γ (RORα and RORγ) and/or a plasminogen activator inhibitor-1 or variant thereof.

In certain embodiments, the methods further comprise administering a sphingosine-1-phoshate signaling, an inhibitor of retinoic-acid-receptor-related orphan receptors α or γ, and/or an inhibitor of tPA microglial activation in combination with induced hypothermia. Typically, therapeutic hypothermia reduces the body temperature of the neonate to below 35 or 34 degrees Celsius and is continued for more than 6 hours, 24 hours, or two days.

In certain embodiments, the subject is a neonate and the mother is diagnosed with a bacterial or viral infection such as chorioamnionitis during or within one or two months of birth.

In certain embodiments, the disclosure relates to methods of preventing hypoxic ischemic encephalopathy in a neonate comprising administering an effective an inhibitor of sphingosine-1-phoshate signaling, an inhibitor of retinoic-acid-receptor-related orphan receptors α or γ, and/or an inhibitor of tPA microglial activation to a pregnant mother.

In certain embodiments, the disclosure relates to methods of preventing hypoxic ischemic encephalopathy in a neonate comprising administering an effective amount sphingosine-1-phoshate signaling, an inhibitor of retinoic-acid-receptor-related orphan receptors α or γ, and/or an inhibitor of tPA microglial activation to a pregnant mother.

In certain embodiments, the disclosure contemplates that administering an effective amount of an inhibitor of sphingosine-1-phoshate signaling, an inhibitor of retinoic-acid-receptor-related orphan receptors a or y, and/or an inhibitor of tPA microglial activation to a subject may be useful before or after cardia arrest or a cardiac surgery in which therapeutic hypothermia is performed to treat or prevent brain injury, e.g., post-cardiac arrest brain injury.

### Formulations

Pharmaceutical compositions disclosed herein can be in the form of pharmaceutically acceptable salts, as generally described below. Some preferred, but non-limiting examples of suitable pharmaceutically acceptable organic and/or inorganic acids are hydrochloric acid, hydrobromic acid, sulfuric acid, nitric acid, acetic acid and citric acid, as well as other pharmaceutically acceptable acids known per se (for which reference is made to the references referred to below).

When the compounds of the disclosure contain an acidic group as well as a basic group, the compounds of the disclosure can also form internal salts, and such compounds are within the scope of the disclosure. When a compound contains a hydrogen-donating heteroatom (e.g. NH), salts are contemplated to cover isomers formed by transfer of the hydrogen atom to a basic group or atom within the molecule.

Pharmaceutically acceptable salts of the compounds include the acid addition and base salts thereof. Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases can also be formed, for example, hemisulphate and hemicalcium salts. For a review on suitable salts, see Handbook of Pharmaceutical Salts: Properties, Selection, and Use by Stahl and Wermuth (Wiley-VCH, 2002), incorporated herein by reference.

The compounds described herein can be administered in the form of prodrugs. A prodrug can include a covalently bonded carrier which releases the active parent drug when administered to a mammalian subject. Prodrugs can be prepared by modifying functional groups present in the compounds in such a way that the modifications are cleaved, either in routine manipulation or in vivo, to the parent compounds. Prodrugs include, for example, compounds wherein a hydroxyl group is bonded to any group that, when administered to a mammalian subject, cleaves to form a free hydroxyl group. Examples of prodrugs include, but are not limited to, acetate, formate and benzoate derivatives of alcohol functional groups in the compounds. Examples of structuring a compound as prodrugs can be found in the book of Testa and Caner, Hydrolysis in Drug and Prodrug Metabolism, Wiley (2006) hereby incorporated by reference. Typical prodrugs form the active metabolite by transformation of the prodrug by hydrolytic enzymes, the hydrolysis of amides, lactams, peptides, carboxylic acid esters, epoxides or the cleavage of esters of inorganic acids.

Pharmaceutical compositions typically comprise an effective amount of a compound and a suitable pharmaceutical acceptable carrier. The preparations can be prepared in a manner known per se, which usually involves mixing the at least one compound according to the disclosure with the one or more pharmaceutically acceptable carriers, and, if desired, in combination with other pharmaceutical active compounds, when necessary under aseptic conditions. Reference is made to U.S. Pat. No. 6,372,778, U.S. Pat. No. 6,369,086, U.S. Pat. No. 6,369,087 and U.S. Pat. No. 6,372,733 and the further references mentioned above, as well as to the standard handbooks, such as the latest edition of Remington's Pharmaceutical Sciences. It is well known that ester prodrugs are readily degraded in the body to release the corresponding alcohol. See e.g., Imai, Drug Metab Pharmacokinet. (2006) 21(3):173-85, entitled “Human carboxylesterase isozymes: catalytic properties and rational drug design.”

Generally, for pharmaceutical use, the compounds can be formulated as a pharmaceutical preparation comprising at least one compound and at least one pharmaceutically acceptable carrier, diluent or excipient and/or adjuvant, and optionally one or more further pharmaceutically active compounds.

The pharmaceutical preparations of the disclosure are preferably in a unit dosage form, and can be suitably packaged, for example in a box, blister, vial, bottle, sachet, ampoule or in any other suitable single-dose or multi-dose holder or container (which can be properly labeled); optionally with one or more leaflets containing product information and/or instructions for use. Generally, such unit dosages will contain between 1 and 1000 mg, and usually between 5 and 500 mg, of the at least one compound of the disclosure e.g., about 10, 25, 50, 100, 200, 300 or 400 mg per unit dosage.

The compounds can be administered by a variety of routes including the oral, ocular, rectal, transdermal, subcutaneous, intravenous, intramuscular or intranasal routes, depending mainly on the specific preparation used. The compound will generally be administered in an “effective amount,” by which it is meant any amount of a compound that, upon suitable administration, is sufficient to achieve the desired therapeutic or prophylactic effect in the subject to which it is administered. Usually, depending on the condition to be prevented or treated and the route of administration, such an effective amount will usually be between 0.01 to 1000 mg per kilogram body weight of the patient per day, more often between 0.1 and 500 mg, such as between 1 and 250 mg, for example about 5, 10, 20, 50, 100, 150, 200 or 250 mg, per kilogram body weight of the patient per day, which can be administered as a single daily dose, divided over one or more daily doses. The amount(s) to be administered, the route of administration and the further treatment regimen can be determined by the treating clinician, depending on factors such as the age, gender and general condition of the patient and the nature and severity of the disease/symptoms to be treated. Reference is made to U.S. Pat. No. 6,372,778, U.S. Pat. No. 6,369,086, U.S. Pat. No. 6,369,087 and U.S. Pat. No. 6,372,733 and the further references mentioned above, as well as to the standard handbooks, such as the latest edition of Remington's Pharmaceutical Sciences.

Formulations containing one or more of the compounds described herein can be prepared using a pharmaceutically acceptable carrier composed of materials that are considered safe and effective and can be administered to an individual without causing undesirable biological side effects or unwanted interactions. The carrier is all components present in the pharmaceutical formulation other than the active ingredient or ingredients. As generally used herein “carrier” includes, but is not limited to, diluents, binders, lubricants, disintegrators, fillers, pH modifying agents, preservatives, antioxidants, solubility enhancers, and coating compositions.

Carrier also includes all components of the coating composition which can include plasticizers, pigments, colorants, stabilizing agents, and glidants. Delayed release, extended release, and/or pulsatile release dosage formulations can be prepared as described in standard references such as “Pharmaceutical dosage form tablets,” eds. Liberman et. al. (New York, Marcel Dekker, Inc., 1989), “Remington-The science and practice of pharmacy,” 20th ed., Lippincott Williams & Wilkins, Baltimore, Md., 2000, and “Pharmaceutical dosage forms and drug delivery systems,” 6th Edition, Ansel et al., (Media, Pa.: Williams and Wilkins, 1995). These references provide information on carriers, materials, equipment and process for preparing tablets and capsules and delayed release dosage forms of tablets, capsules, and granules.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material can contain conventional carriers such as plasticizers, pigments, colorants, glidants, stabilization agents, pore formers and surfactants.

Optional pharmaceutically acceptable excipients present in the drug-containing tablets, beads, granules or particles include, but are not limited to, diluents, binders, lubricants, disintegrants, colorants, stabilizers, and surfactants.

Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

Surfactants can be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-beta-alanine, sodium N-lauryl-beta-iminodipropionate, myristoamphoacetate, lauryl betaine, and lauryl sulfobetaine.

If desired, the tablets, beads, granules, or particles can also contain minor amount of nontoxic auxiliary substances such as wetting or emulsifying agents, dyes, pH buffering agents, or preservatives.

The compositions described herein can be formulation for modified or controlled release. Examples of controlled release dosage forms include extended release dosage forms, delayed release dosage forms, pulsatile release dosage forms, and combinations thereof.

The extended release formulations are generally prepared as diffusion or osmotic systems, for example, as described in “Remington-The science and practice of pharmacy” (20th ed., Lippincott Williams & Wilkins, Baltimore, Md., 2000). A diffusion system typically consists of two types of devices, a reservoir and a matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, cellulosic polymers such as methyl and ethyl cellulose, hydroxyalkylcelluloses such as hydroxypropyl-cellulose, hydroxypropylmethylcellulose, sodium carboxymethylcellulose, and Carbopol® 934, polyethylene oxides and mixtures thereof. Fatty compounds include, but are not limited to, various waxes such as carnauba wax and glyceryl tristearate and wax-type substances including hydrogenated castor oil or hydrogenated vegetable oil, or mixtures thereof.

In certain preferred embodiments, the plastic material is a pharmaceutically acceptable acrylic polymer, including but not limited to, acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer poly(methyl methacrylate), poly(methacrylic acid)(anhydride), polymethacrylate, polyacrylamide, poly(methacrylic acid anhydride), and glycidyl methacrylate copolymers.

In certain preferred embodiments, the acrylic polymer is comprised of one or more ammonio methacrylate copolymers. Ammonio methacrylate copolymers are well known in the art, and are described in NF XVII as fully polymerized copolymers of acrylic and methacrylic acid esters with a low content of quaternary ammonium groups. In one preferred embodiment, the acrylic polymer is an acrylic resin lacquer such as that which is commercially available from Rohm Pharma under the tradename Eudragit®. In further preferred embodiments, the acrylic polymer comprises a mixture of two acrylic resin lacquers commercially available from Rohm Pharma under the tradenames Eudragit® RL3OD and Eudragit ® RS30D, respectively. Eudragit® RL30D and Eudragit® RS30D are copolymers of acrylic and methacrylic esters with a low content of quaternary ammonium groups, the molar ratio of ammonium groups to the remaining neutral (meth)acrylic esters being 1:20 in Eudragit® RL30D and 1:40 in Eudragit® RS30D. The mean molecular weight is about 150,000. Edragit® S-100 and Eudragit® L-100 are also preferred. The code designations RL (high permeability) and RS (low permeability) refer to the permeability properties of these agents. Eudragit® RL/RS mixtures are insoluble in water and in digestive fluids. However, multiparticulate systems formed to include the same are swellable and permeable in aqueous solutions and digestive fluids.

The polymers described above such as Eudragit® RL/RS can be mixed together in any desired ratio in order to ultimately obtain a sustained-release formulation having a desirable dissolution profile. Desirable sustained-release multiparticulate systems can be obtained, for instance, from 100% Eudragit® RL, 50% Eudragit® RL and 50% Eudragit® RS, and 10% Eudragit® RL and 90% Eudragit® RS. One skilled in the art will recognize that other acrylic polymers can also be used, such as, for example, Eudragit® L.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above can be combined in a final dosage form comprising single or multiple units. Examples of multiple units include, but are not limited to, multilayer tablets and capsules containing tablets, beads, or granules. An immediate release portion can be added to the extended release system by means of either applying an immediate release layer on top of the extended release core using a coating or compression process or in a multiple unit system such as a capsule containing extended and immediate release beads.

Extended release tablets containing hydrophilic polymers are prepared by techniques commonly known in the art such as direct compression, wet granulation, or dry granulation. Their formulations usually incorporate polymers, diluents, binders, and lubricants as well as the active pharmaceutical ingredient. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In the congealing method, the drug is mixed with a wax material and either spray-congealed or congealed and screened and processed. Delayed release formulations are created by coating a solid dosage form with a polymer film, which is insoluble in the acidic environment of the stomach, and soluble in the neutral environment of the small intestine.

The delayed release dosage units can be prepared, for example, by coating a drug or a drug-containing composition with a selected coating material. The drug-containing composition can be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and can be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit® (Rohm Pharma; Westerstadt, Germany), including Eudragit® L30D-55 and L100-55 (soluble at pH 5.5 and above), Eudragit® L-100 (soluble at pH 6.0 and above), Eudragit® S (soluble at pH 7.0 and above, as a result of a higher degree of esterification), and Eudragits® NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials can also be used. Multi-layer coatings using different polymers can also be applied.

The preferred coating weights for particular coating materials can be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition can include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates can also be used. Pigments such as titanium dioxide can also be used. Small quantities of an anti-foaming agent, such as a silicone (e.g., simethicone), can also be added to the coating composition.

Alternatively, each dosage unit in the capsule can comprise a plurality of drug-containing beads, granules or particles. As is known in the art, drug-containing “beads” refer to beads made with drug and one or more excipients or polymers. Drug-containing beads can be produced by applying drug to an inert support, e.g., inert sugar beads coated with drug or by creating a “core” comprising both drug and one or more excipients. As is also known, drug-containing “granules” and “particles” comprise drug particles that can or cannot include one or more additional excipients or polymers. In contrast to drug-containing beads, granules and particles do not contain an inert support. Granules generally comprise drug particles and require further processing. Generally, particles are smaller than granules, and are not further processed. Although beads, granules and particles can be formulated to provide immediate release, beads and granules are generally employed to provide delayed release.

### EXAMPLES

Inflammation-sensitized HI Induces Systemic T_{H}17-ike Cells and Focal Invasion of CD43+ Leukocytes through the Choroid Plexus

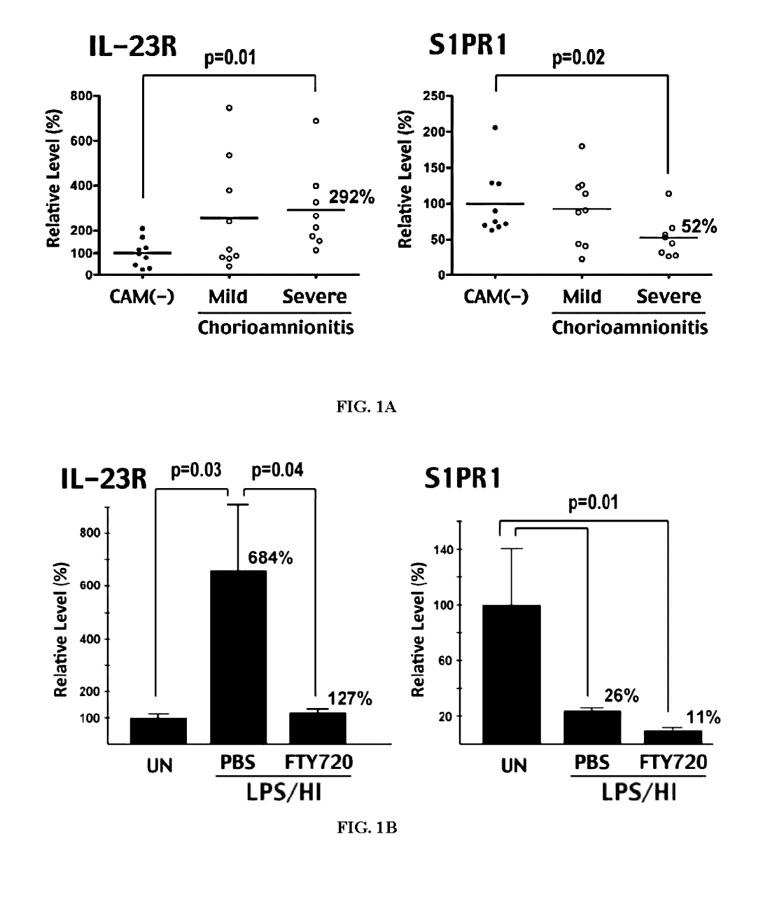

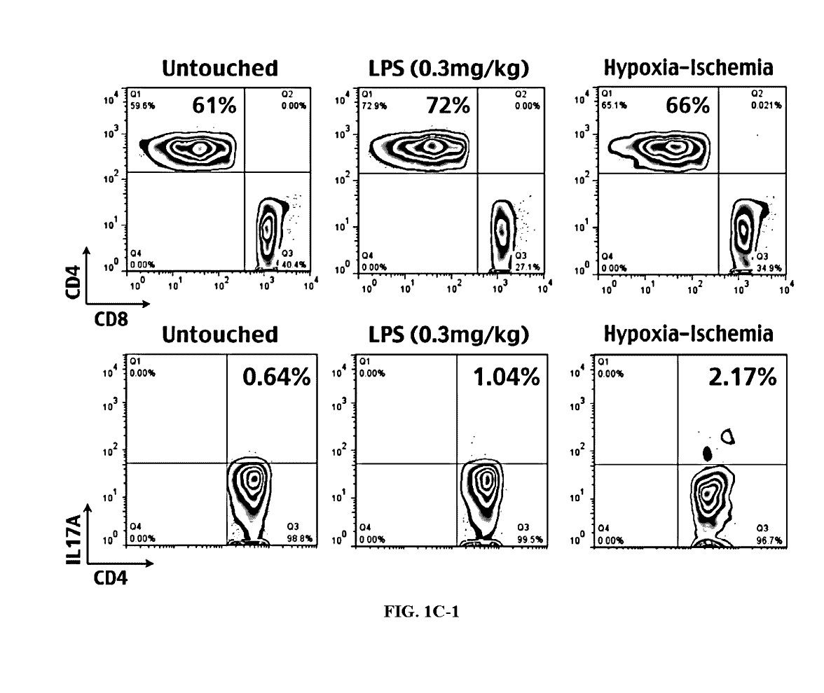

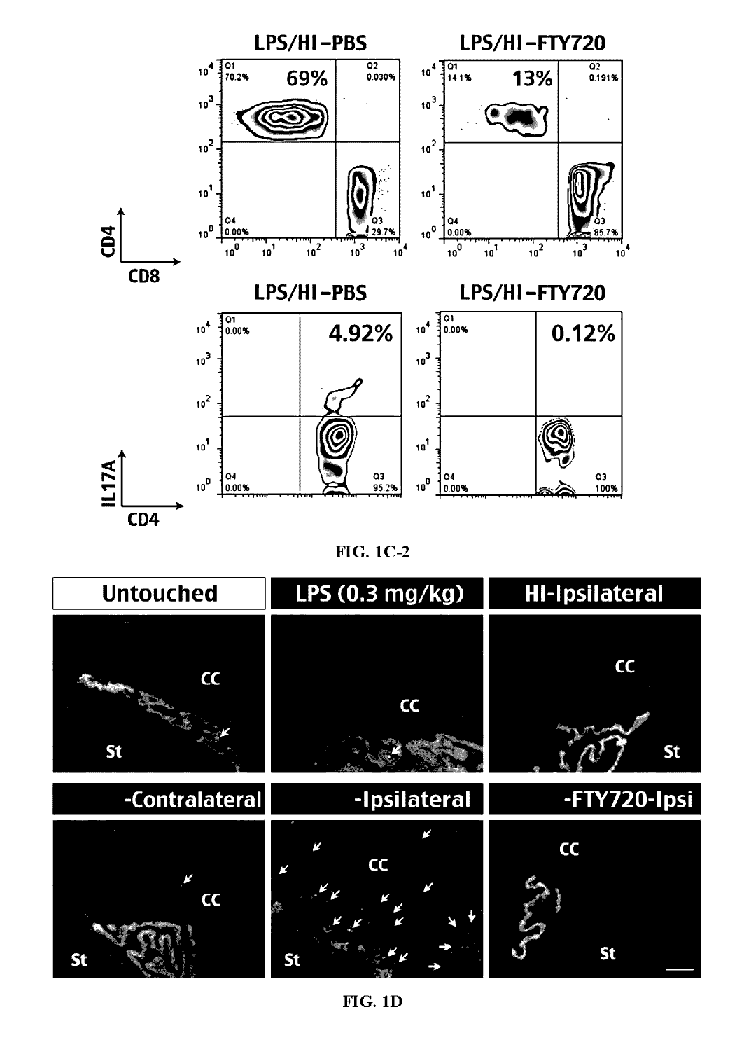

To test whether intrauterine infection accelerates the development of T_{H}17 lymphocytes, the expression of IL-23 receptor (IL-23R) mRNA, a marker of T_{H}17 cells, was compared in aliquots of cord blood mononuclear cells isolated from late preterm neonates (32-36 gestational weeks) with and without histological chorioamnionitis. This analysis showed 292% increase of IL-23R mRNA in those with severe chorioamnionitis (fetal vasculitis) (FIG. 1A). A well-characterized LPS-sensitized HI brain injury model in P7 rats was used to compare the effects of low-dose LPS (0.3 mg/kg, IP), HI, and combined LPS/HI on the expression of IL-23R mRNA in the peripheral blood mononuclear cells. LPS-sensitized HI injury caused significant increase of IL-23R mRNA expression at 4 h after insult, while pure-HI and low-dose LPS challenge lacked this effect.

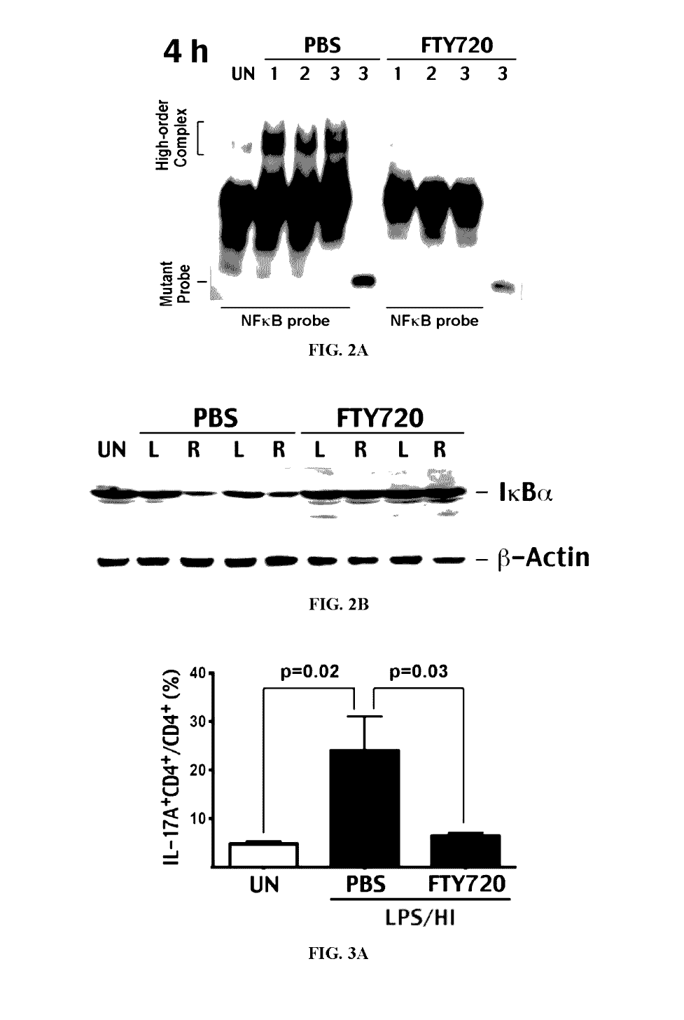

The expression of IL-17A mRNA, however, was not significantly elevated in either human or rodent mononuclear cells, suggesting that the IL-23R+ mononuclear cells are early-stage T_{H}17 cells, but not fully matured T_{H}17 effector cells. Consistent with this idea, flow cytometry analysis showed a comparable CD4+ to CD8+ T-cell ratio in the blood mononuclear cells among untouched and low-dose LPS-, HI-, or LPS/HI-injured rat pups, and a small increase of CD4+ IL-17A+ lymphocytes (4.92%) at 16 h recovery after the dual LPS/HI-insult (FIG. 1C-1 and FIG. 1C-2). These results suggest that LPS-sensitized neonatal HI injury does not induce a systemic surge of mature T_{H}17 effector cells.

The possibility of early influx of lymphocytes into neonatal brains following various stimuli was tested by immunostaining with the anti-CD43 (leukosialin) antibody, a commonly used pan-T-cell marker that is expressed to a lesser degree in other leukocytes. The choroid plexus in untouched rats displays a high level of CD43-immunoreactivity among cell-junctions, likely as part of the dense glycocalyx oligosaccharide domain in the brain-interstitial fluid interface due to its >100 extracellular sialic acid residues (FIG. 1D). The anti-CD43 immunoreactivity in the choroid plexus was reduced after exposure to low-dose LPS, and further decreased after pure- and LPS-sensitized HI injury at 4 h recovery (FIG. 1D). Contrary to the normal ribbon-like CD43-immunoreactivity, many CD43+ cells were detected in the ipsilateral choroid plexus and scattered into the nearby white matter after the dual LPS/HI injury (FIG. 1D). In the contralateral choroid plexus after LPS/HI injury, fewer CD43+ cells were present and they were imbedded in the preserved CD43+ strip. These results suggest that HI brain injury down-regulates CD43-immunoreactivity in the choroid plexus, and the dual LPS/HI insult causes early influx of CD43+ leukocytes into neonatal brains.

### Systemic FTY720 Administration Prevents Early Infiltration of CD43+ Leukocytes

The effects of FTY720 administration (1 μg/g, IP) after LPS/HI insult in P7 rat pups were examined. Consistent with its ability to prevent lymphocyte egress from lymph nodes, FTY720 blocked the increase of IL-23R mRNAs in the rat blood mononuclear cells at 4 h recovery (FIG. 1B). Moreover, FTY720 preferentially reduced CD4+ T-cells and produced near-complete depletion of systemic CD4+ IL-17A+ T-lymphocytes at 16 h after LPS/HI injury (FIG. 1C-1 and FIG. 1C-2). Finally, the FTY720 treatment preserved the ribbon-like CD43 immunoreactivity in the ipsilateral choroid plexus and mitigated the influx of CD43+ leukocytes at 4 h after the dual LPS/HI injury (FIG. 1D).

### CD43+ Leukocytes Enter Neonatal Brains in Two Distinct Sequences Following LPS/HI Injury

In experimental autoimmune encephalomyelitis (EAE), T_{H}17 cells enter the CNS initially through the choroid plexus and secondarily in the leptomeningeal space after substantial brain damage and opening of the BBB. Similarly, at 4 h following the LPS/HI injury, CD43+ leukocytes were only detected in the choroid plexus and the nearby subcortical WM, where they were mixed with Iba1+ amoeboid microglial cells. In contrast, very few CD43+ cells were present in the leptomeningeal space at this early time-point. Post-LPS/HI administration of FTY720 led to the absence of CD43+ cells in the subcortical WM. The FTY720 treatment also greatly reduced the LPS/HI-triggered nuclear NF-κB activity and degradation of cytosolic IKBa at 4 h recovery (FIG. 2A, B), which are among the earliest biochemical signs for neuroinflammatory response in this model.

At 18-24 h after LPS/HI injury, there were more evidences of neuroinflammation. Meanwhile, the leptomeningeal space became dilated and filled with CD43+ leukocytes in the ipsilateral, but not the contralateral hemisphere, or after the FTY720 treatment. Similarly, the LPS/HI-injured cerebral cortex contained a large number of CD43+ leukocytes and numerous round, Iba1+ phagocytic microglia/macrophages, which were both absent in the contralateral hemisphere and after the FTY720 treatment. These observations suggest two spatiotemporally distinct routes of leukocyte influx in LPS/HI-injured neonatal brains, which were both greatly attenuated by post-injury administration of FTY720.

### FTY720 Attenuates Neuroinflammation and Reduces CD4+ IL-17A+ Lymphocytes Following LPS/HI Injury.

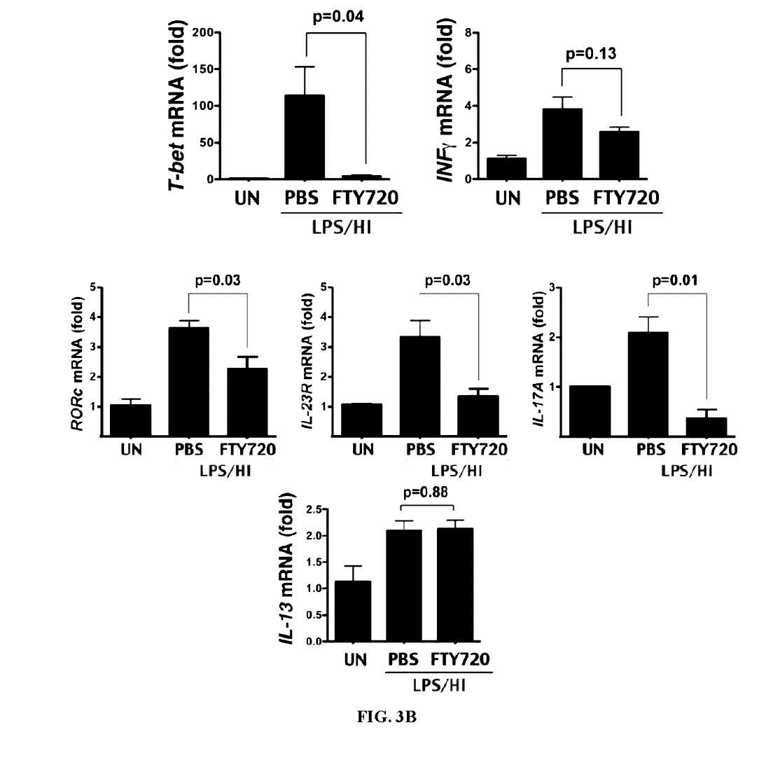

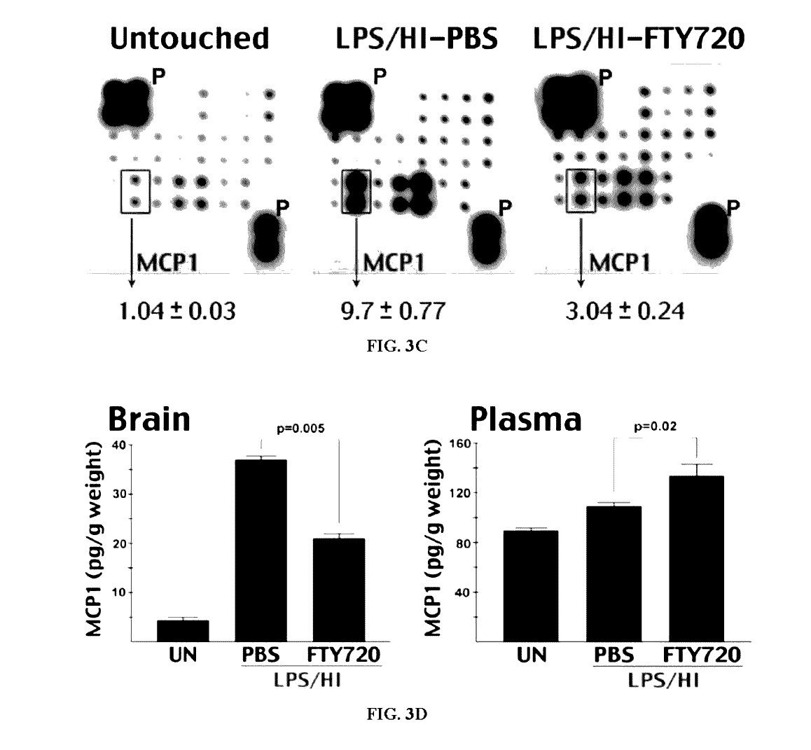

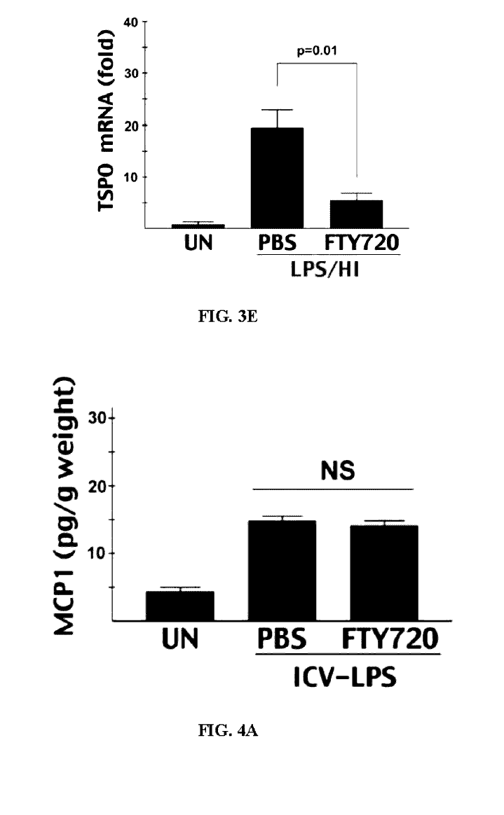

Flow cytometry we used to compare the percentage of CD4+ IL-17A+T-cells at 24 h after LPS, HI (90 min), or LPS+80 min HI brain injury. A longer duration of pure-HI insult was chosen because 80-min HI produced milder brain damage and little neuroinflammation. The T_{H}17/IL-17 activity was compared under pure-HI (90 min) and LPS/HI (80 min) insults that produce similar levels of brain injury. The experiments showed <3% of CD4+ IL-17A+lymphocytes among CD4+ T-cells in untouched, LPS-challenged, and pure-HI injured brains. The dual LPS/HI insult led to a significant increase to 21% of CD4+ IL-17A+ T-cells, which was reduced to 12% by FTY720 treatment (FIG. 3A).

RT-PCR was used to compare the mRNA levels of RORc, IL-23R, IL-17A, IL-22 (all markers of T_{H}17 cells), Tbx21/T-bet (a marker of T_{H}1 and mixed T_{H}1/T_{H}17 cells), Interferon-γ (a T_{H}1 cell marker), as well as Gata3 and IL-13 (markers of T_{H}2 cells) between pure-HI injury and LPS/HI injury with or without the FTY720 treatment. The analysis showed that combined LPS/HI insult has a greater propensity than pure-HI injury to induce T_{H}17 markers, which were markedly reduced by the FTY720 treatment (p<0.05-difference in the induction of RORc, IL-23R, and IL-17A mRNA between HI and LPS/HI injury and the amelioration by FTY720). In contrast, the enhancement of T_{H}1 and T_{H}2 markers by the LPS/HI injury was either insignificant (in Tbx21, Gata3, IL-13 mRNA) or reversed by the pure-HI insult (in Interferon-γ). Further, the FTY720 treatment only significantly attenuated the induction of Tbx21 mRNA, but not the other T_{H}1 and T_{H}2 markers (FIG. 3B). Together, the flow cytometry and RT-PCR findings suggest that combined LPS/HI injury induces, while FTY720 attenuates, the T_{H}17/IL-17A-mediated immune response in neonatal brains.

In addition, the FTY720 treatment markedly reduced the expression of MCP 1 (Monocyte Chemoattractant Protein 1) protein and TSPO (Translocator Protein; Peripheral Benzodiazepine Receptor), which are both indicators of neuroinflammation and microglial activation at 18-24 h following LPS/HI injury (FIG. 3C-E). In contrast, the FTY720 treatment failed to prevent the LPS/HI-triggered rise of plasma MCP1, supporting its CNS-centric effects (FIG. 3E).

### FTY720 Lacks Direct Inhibitory Effects on Microglia

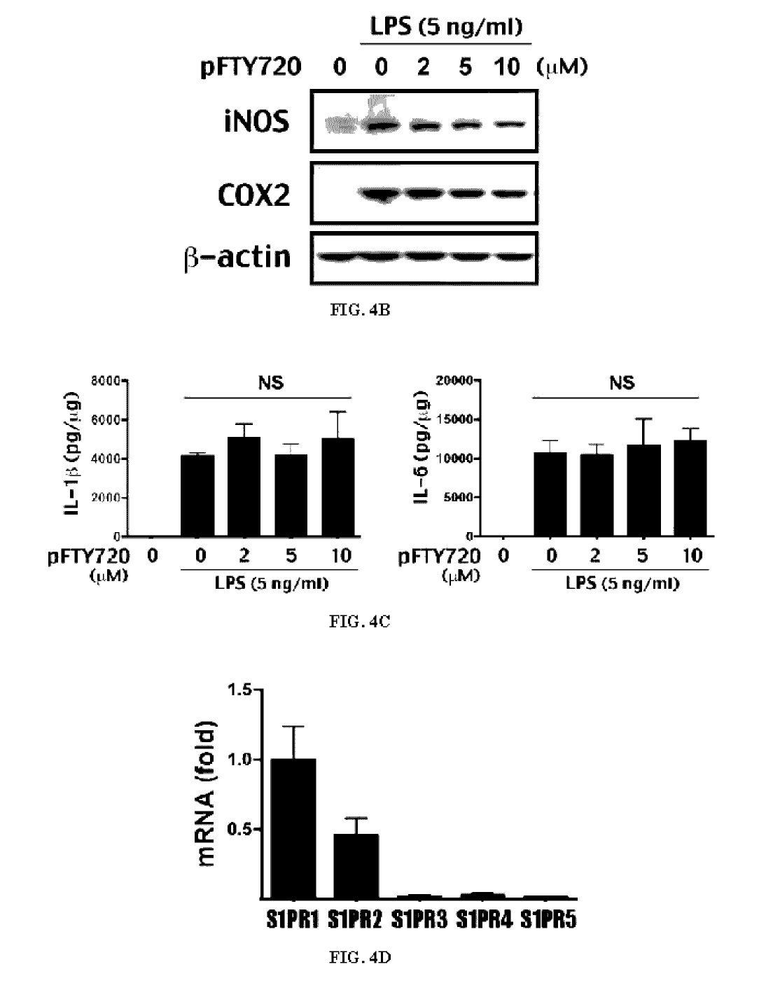

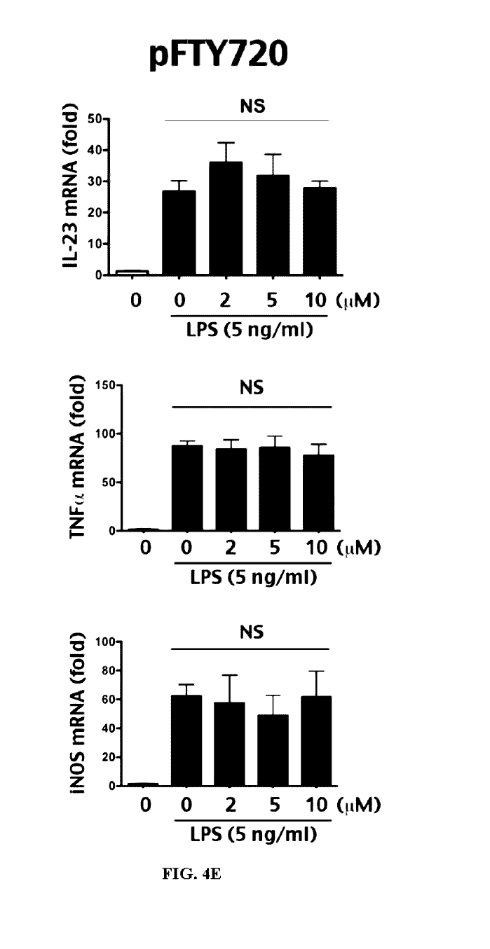

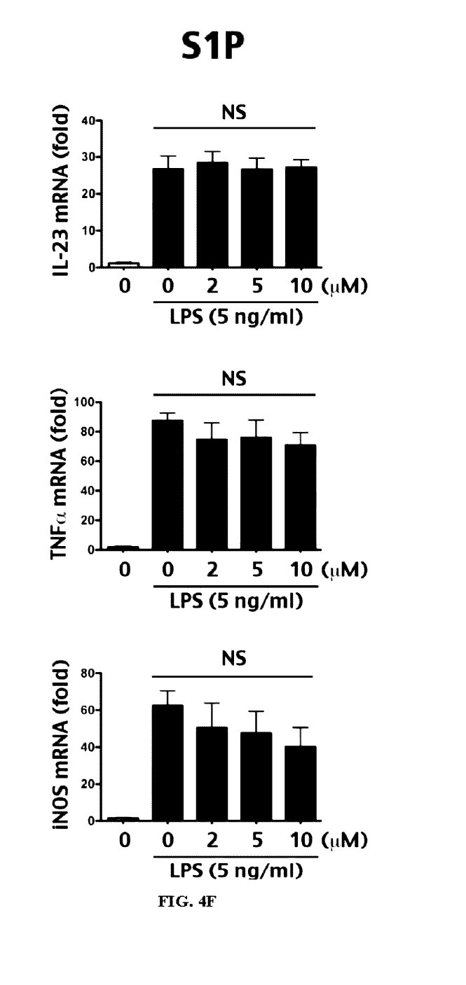

Experiments herein indicate FTY720 primarily blocks brain influx of leukocytes to prevent LPS/HI-induced neuroinflammation. An alternative explanation is that FTY720 might directly inhibit microglia activation. To distinguish between these two scenarios, the effects of FTY720 was tested on the MCP1 induction following cerebral ventricle-injection of LPS, a paradigm of direct microglial activation. This experiment showed that the FTY720 treatment was unable to prevent direct LPS-induced MCP1 induction (FIG. 4A). Intracerebroventricular (ICV) injection of LPS was performed in bitransgenic CX3CR1-GFP; CCR2-RFP mice, in which the microglia and circulating monocytes are marked by the green and red fluorescence protein, respectively. In unchallenged mice, the brains were largely devoid of monocytes and the microglial processes were excluded from the hippocampal pyramidal cell layer. In contrast, after ICV-LPS injection, both saline- and FTY720-treated mice showed intrusion of hypertrophic microglia processes in the hippocampal pyramidal cell layer and a massive influx of monocytes, by the increase of brain MCP1 level (FIG. 4B). Likewise, in immortalized microglial cells, low-dose LPS (5 ng/ml) readily induced the expression of iNOS (inducible Nitric Oxide Synthase), COX2 (Cyclooxygenase 2), and pro-inflammatory IL-1β or IL-6 cytokines despite the presence of up to 10 μM phosphorylated-FTY720 (pFTY720) (FIG. 4B-C).

These in-vivo and in-vitro results argued against direct inhibition of microglia by FTY720, which prompted us to examine the expression of S1P receptor isoforms in neonatal rat microglia. The RT-qPCR analysis showed that, while S1PR1 is the most abundant isoform in microglia, there was also a high expression level of S1PR2, an isoform that pFTY720 lacks binding affinity to, which could render (p)FTY720 unable to prevent microglia activation directly (FIG. 4E). Thus, the effects of pFTY720 and S1P on LPS-induced expression of IL-23 (a key factor for T_{H}17 cell differentiation), TNFα (Tumor Necrosis Factor-alpha), and iNOS mRNA were compared. This analysis showed that neither pFTY720 nor S1P (up to 10 μM) suppressed microglial activation directly (FIG. 4F). Finally, the effects of S1P and Tat-NBD, an anti-NFKB peptide, were compared on the intracellular signaling responses to LPS in immortalized microglial cells. Tat-NBD provided dose-dependent attenuation of LPS-induced degradation of cytosolic IκBα and accumulation of nuclear NFκB/Re1A, while up to 20 μM S1P exerted no opposing effects (FIG. 4G). In contrast, the S 1P treatment showed dose-dependent reduction of S1PR1 mRNA as negative feedback of the signaling pathway

Together, these in-vivo and in-vitro results suggest that FTY720 (and the active metabolite pFTY720) lack direct inhibition on microglia. Thus, the suppression of microglial activation in neonatal LPS/HI injury is more likely mediated through another population of target cells.

FTY720 Selectively Prevents LPS-sensitized, but not pure-HI Brain Injury

Whether (p)FTY720 exerts direct neuroprotection was investigated. The effects of FTY720 on pure-HI (90 min) and LPS-sensitized HI insult (80 min) were compared reasoning that FTY720 should oppose both insults if its benefits are derived primarily from neuronal protection. Of note, because 80 min pure-HI insult caused little brain damage, a longer duration of pure-HI (90 min) we used in this experiment. FTY720 markedly attenuated LPS/HI-triggered BBB damage, as shown by reduced NaF extravasation and diminished MMP9 (matrix metalloproteinase-9) activity in the ipsilateral hemisphere (FIG.

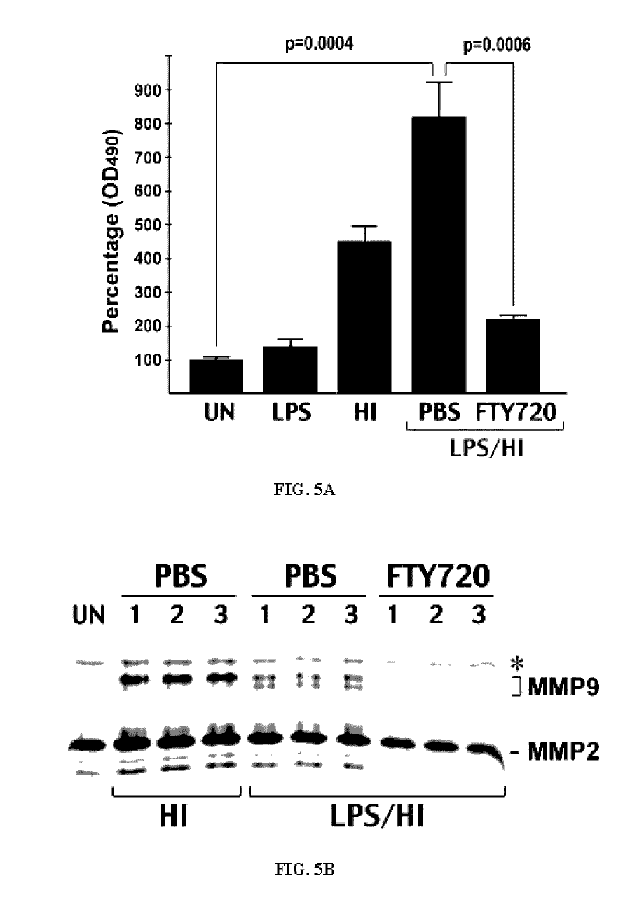

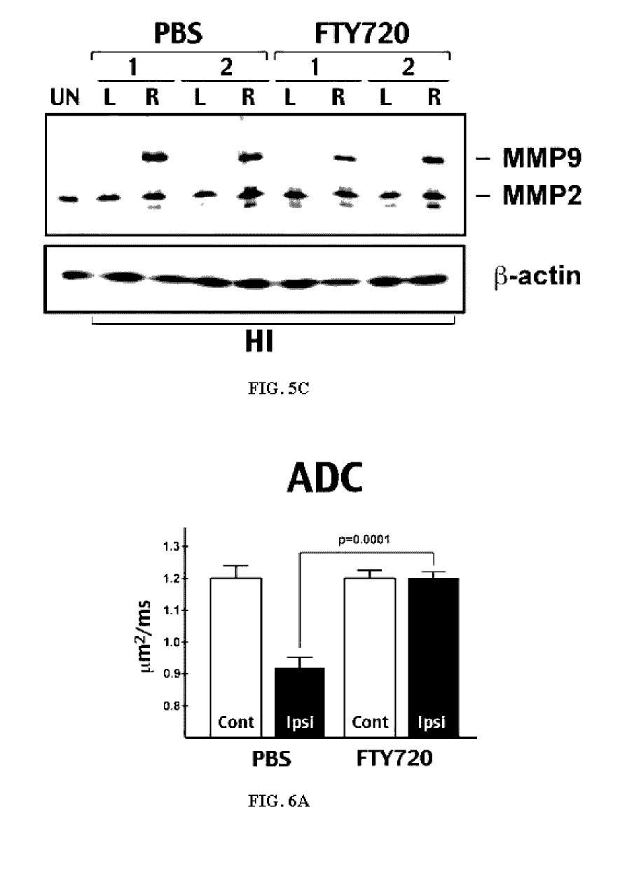

5A-E). This experiment also showed greater BBB damage and more advanced MMP9 processing by the LPS/HI insults. Moreover, FTY720 provided dose-dependent reduction of brain atrophy, salvaging >90% of LPS/HI-induced tissue loss in the hippocampus and the cerebral cortex if the treatment was started within 30 min after injury (FIG. 5C. In contrast, the acute FTY720 treatment failed to prevent MMP9 activation or brain atrophy after pure-HI injury (FIG. 5C).

### FTY720 Reduces Acute Brain Atrophy and Protects Normal Development of Motor Functions

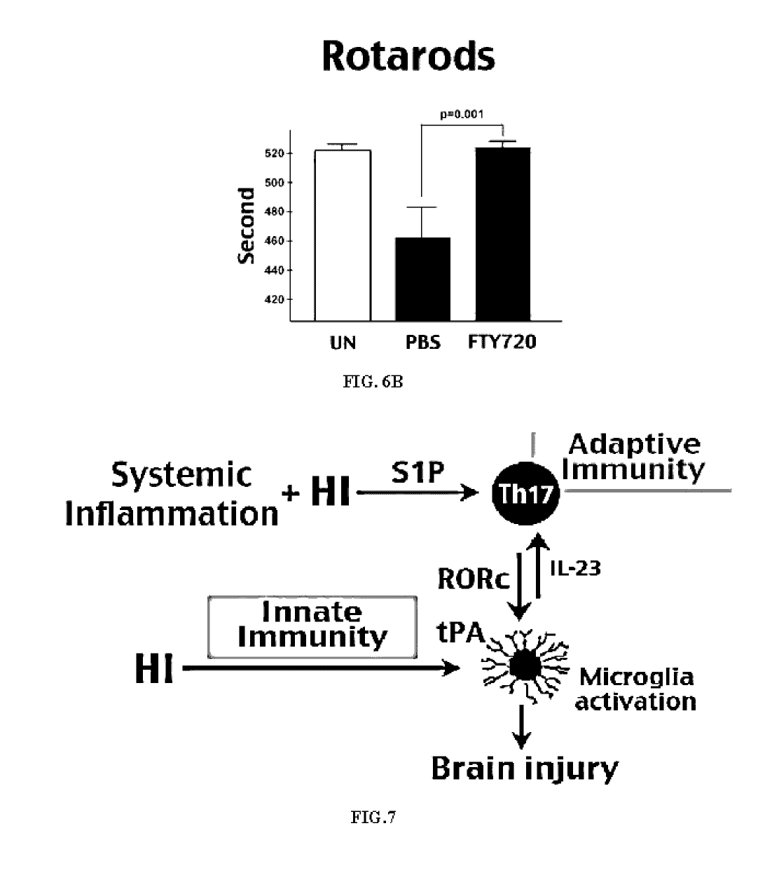

Magnetic resonance imaging (MRI) and behavioral testing were used to assess both acute and long-term effects of FTY720 treatment in LPS/HI injury. Manganese-enhanced MRI (MEMRI) showed that FTY720 preserved brain cytoarchitectural distinctions in the ipsilateral hemisphere, which became obfuscated in saline-treated animals at 24 h post-LPS/HI. Moreover, while saline-treated pups showed reduction of apparent diffusion coefficient (ADC) coinciding with increased sodium concentration in the ipsilateral hemisphere at 24 h recovery, the FTY720 treatment mitigated both responses (FIG. 6A). In addition, in-vivo diffusion tensor imaging (DTI) and tract-based spatial statistics (TBSS) analysis identified areas of p<0.05-reduction in mean diffusivity (in gray matter) or fractional anisotropy (in white matter) at 24 h recovery in the ipsilateral hemisphere of saline-treated, but not FTY720-treated animals. Two weeks later, the TBSS-identified at-risk gray and white matter became atrophic or distorted in saline-treated rat pups. Finally, the FTY720-treated rats showed near-normal gain in body weight and the latency in rotarods by 24 days of age, far better than the outcomes in saline-treated siblings (FIG. 6B).

Together, these results suggest that the FTY720 treatment not only reduces acute brain atrophy, but also protects normal growth and motor development following neonatal LPS/HI brain injury.

### Systemic Administration of SR1001, a RORc-inhibitor, Reduces LPS/HI Brain Injury

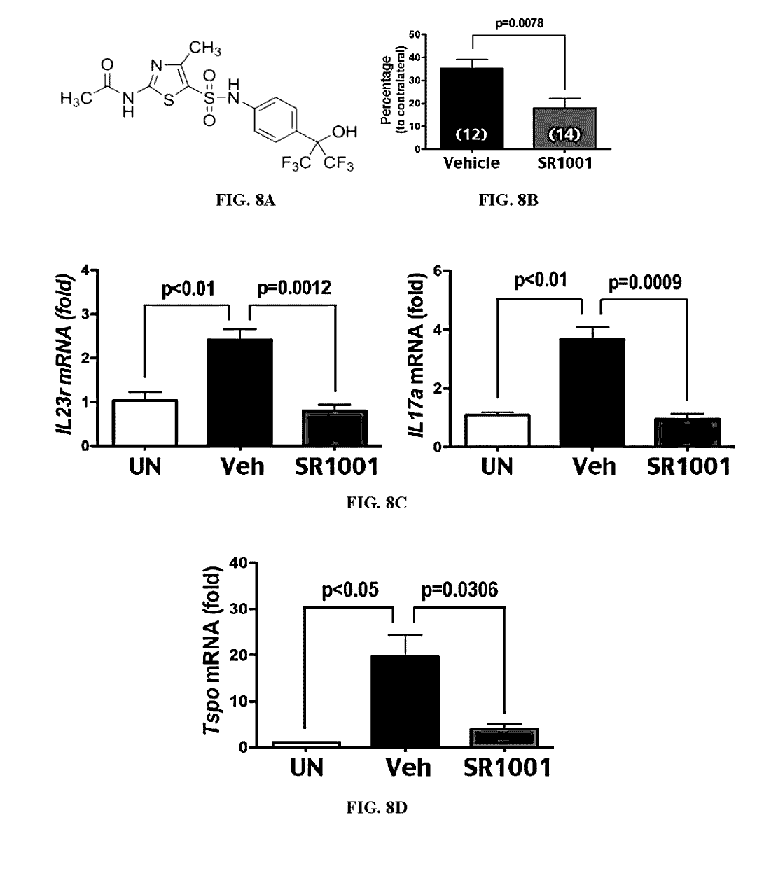

An experiment was performed to test the effects of SR1001, a small-molecule inhibitor of RORc, in LPS/HI injury in P10 mice. Two doses of 25 mg/kg SR1001 (16 h pre- & 1 h post-LPS/HI) significantly reduced brain atrophy (n=12-14), the induction of T_{H}17 cells and microglial activation/TSPO marker (FIG. 8B-D). This data confirms the benefits of RORc inhibitors against neonatal infection/HI injury.

What is claimed:

1. A method of treating or preventing brain injury comprising administering an effective amount or fingolimod (FTY720 or 2-amino-2-[2-(4-octylphenyl)ethyl]propane-1,3-diol), or salt or derivative thereof to a subject in need thereof.

2. The method of claim 1, wherein the subject is a neonate or a neonate born prematurely.

3. The method of claim 2, wherein neonate is born more than one, two, three, or four weeks, premature.

4. The method of claim 1, further comprising administering fingolimod in combination with an inhibitor of retinoic-acid-receptor-related orphan receptors α or γt (RORα and RORγt) or plasminogen activator inhibitor-1 or variant thereof.

5. The method of claim 4, wherein the inhibitor of retinoic-acid-receptor-related orphan receptors α or γt is N-(5-(N-(4-(1,1,1,3,3,3-Hexafluoro-2-hydroxypropan-2-yl)phenyl)sulphamoyl)-4-methylthiazol-2-yl)acetamide (SR1001) or salt or derivative thereof.

6. The method of claim 1, further comprising administering fingolimod in combination with therapeutic hypothermia.

7. The method of claim 6, wherein therapeutic hypothermia is reduces the body temperature of the neonate to below 35 or 34 degrees Celsius.

8. The method of claim 6, wherein the therapeutic hypothermia is continued for more than 6 hours, 24 hours, or two days.

9. The method of claim 1, wherein the subject is a neonate and the mother is diagnosed with chorioamnionitis during or within one or two months of birth.

10. A method of treating or preventing brain injury comprising administering an inhibitor of retinoic-acid-receptor-related orphan receptors α or γt (RORα and RORγt) to a subject in need thereof.

11. The method of claim 10, wherein the inhibitor of retinoic-acid-receptor-related orphan receptors α or γt is N-(5-(N-(4-(1,1,1,3,3,3-Hexafluoro-2-hydroxypropan-2-yl)phenyl)sulphamoyl)-4-methylthiazol-2-yl)acetamide (SR1001), derivative, ester, or salt thereof.

12. The method of claim 10, further comprising administering N-(5-(N-(4-(1,1,1,3,3,3-Hexafluoro-2-hydroxypropan-2-yl)phenyl)sulphamoyl)-4-methylthiazol-2-yl)acetamide (SR1001), derivative, ester, or salt thereof in combination with therapeutic hypothermia.

13. A method of preventing hypoxic ischemic encephalopathy in a neonate comprising administering an effective amount N-(5-(N-(4-(1,1,1,3,3,3-Hexafluoro-2-hydroxypropan-2-yl)phenyl)sulphamoyl)-4-methylthiazol-2-yl)acetamide (SR1001), derivative, ester, or salt thereof to a pregnant mother.

14. A method of preventing hypoxic ischemic encephalopathy in a neonate comprising administering an effective amount or fingolimod (FTY720 or 2-amino-2-[2-(4-octylphenyl)ethyl]propane-1,3-diol), derivative, ester, or salt thereof to a pregnant mother.


SEQUENCE LISTING (1 sequence)

SEQ ID NO: 1 - protein, 379 aa, Artificial Sequence [sequence omitted]